(19) United States
(12) Patent Application Publication
(10) Pub. No.: US 2025/0129395 A1
(43) Pub. Date: Apr. 24, 2025

(54) MEDIA REFINEMENT AND NUTRIENT FEEDING APPROACHES TO INCREASE POLYUNSATURATED FATTY ACID PRODUCTION

(71) Applicant: DSM IP Assets B.V., TE Heerlen (NL)

(72) Inventors: Jose R. GARCIA, Lorton, VA (US); Adam BURJA, Potomac, MD (US); Xiao Daniel DONG, Columbia, MD (US)

(73) Assignee: DSM IP Assets B.V., TE Heerlen (NL)

(21) Appl. No.: 18/833,170

(22) PCT Filed: Jan. 24, 2023
(86) PCT No.: PCT/IB2023/050609

§ 371 Date: Jul. 25, 2024

Related U.S. Application Data
(60) Provisional application No. 63/302,696, filed on Jan. 25, 2022.

(51) Int. Cl. C12P 7/6432; C12P 7/6434

(52) U.S. Cl. CPC C12P 7/6432; C12P 7/6434

(57) ABSTRACT

The present disclosure provides a method for increasing production of a polyunsaturated fatty acid, especially eicosapentaenoic acid (EPA), in a microorganism, comprising adjusting the macro-and/or micronutrients during fermentation. The invention includes any culture, biomass, and oil produced by the method.

### CROSS-REFERENCE TO RELATED APPLICATION

This is an International Application under the Patent Cooperation Treaty. claiming priority to U.S. Provisional Patent Application No. 63/302,696 filed Jan. 25, 2022 the contents of which are incorporated herein by reference in their entirety.

### FIELD OF THE INVENTION

The present disclosure provides a method for increasing production of a poly-unsaturated fatty acid, especially eicosapentaenoic acid (EPA), in a microorganism, comprising refining the micro and/or macronutrients during culture of the microorganism. The invention also is directed to any as well as any culture, biomass, or oil produced by the method.

### BACKGROUND OF THE INVENTION

Polyunsaturated fatty acids (PUFAs) are useful for nutritional applications,

pharmaceutical applications, industrial applications, and other purposes. Polyunsaturated fatty acids (PUFAs) are classified based on the position of the first double bond from the methyl end of the fatty acid: omega-3 (n-3) fatty acids contain a first double bond at the third carbon, while omega-6 (n-6) fatty acids contain a first double bond at the sixth carbon. For example, docosahexaenoic acid (DHA) is an omega-3 long chain polyunsaturated fatty acid (LC-PUFA) with a chain length of 22 carbons and 6 double bonds, often designated as “22:6 n-3.”

Long-chain PUFAs (LC-PUFAs) are fatty acids that contain at least 3 double bonds and have a chain length of 18 or more carbons or 20 or more carbons. The LC-PUFAs of the omega-3 series include, but are not limited to, eicosatrienoic acid (C20:3n-3), eicosatetraenoic acid (C20:4n-3), EPA (C20:5n-3), docosapentaenoic acid (C22:5n-3) (DPA), and DHA (C22:6n-3). LC-PUFAs of the omega-6 series include, but are not limited to, di-homo-gammalinoleic acid (C20:3n-6), arachidonic acid (C20:4n-6) (ARA), docosatetraenoic acid or adrenic acid (C22:4n-6), and docosapentaenoic acid (C22:5n-6) (DPA n-6). The LC-PUFAs also include fatty acids with greater than 22 carbons and 4 or more double bonds including, but not limited to, C24:6 (n-3) and C28:8 (n-3).

LC-PUFAs EPA and DHA are “essential” fatty acids. Because omega-3 fatty acids are not synthesized de novo in the human body, these fatty acids must be derived from nutritional sources.

PUFAs cannot be produced in sufficient amounts for commercial use from fish oil due in part to overfishing. Polyketide synthase-like systems exist in marine bacteria and certain microalgae are capable of synthesizing polyunsaturated fatty acids (PUFAs) from acetyl-CoA and malonyl-CoA anaerobically. Microalgae are a source of PUFAs as they can accumulate lipids up to 80% of their cell dry weight. For example, strains of Thraustochytrid species have been reported to produce omega-3 fatty acids including DHA and EPA as a high percentage, up to 20%, of the total fatty acids produced by the organisms (See U.S. Pat. No. 5,130,242; Huang, J. et al., J. Am. Oil. Chem. Soc. 78:605-610 (2001); Huang, J. et al., Mar. Biotechnol. 5:450-457 (2003)).

Thraustochytrids are microorganisms of the order Thraustochytriales. Thraustochytrids include members of the genus Schizochytrium and Thraustochytrium and have been recognized as an alternative source of omega-3 fatty acids, including DHA and EPA (See U.S. Pat. No. 5,130,242).

High oil production from Schizochytrium, can be obtained as a result of high growth rate by controlling of nutrients such as glucose, nitrogen, sodium and some other environmental factors, such as oxygen concentration, temperature, salinity and pH, achieving high cell densities and DHA productivities. Genetic manipulation of synthetic pathways also has been shown in to increase the yield of EPA in non-thraustochytrid organisms (See Xia et al., Algal Res. 2020; 51; 102038; Adarme-Vega et al., Microbial Cell Factories. 2012; 11:96).

While some organisms are known to natively produce higher e.g., DHA or EPA, controlled manipulation of the ratios of PUFAs, including but not limited to EPA and DHA in strains of microalgae would be beneficial. PUFAs are well known to have cholesterol and hypertension lowering effects that benefit the cardiovascular system. However, the optimal dietary omega-3/omega-6 ratio should be around 1:1-4, but in the Western diet it varies between 1:10 and 1:20. Peltomaa et al., Mar Drugs. 2018 January; 16 (1): 3. An imbalance of dietary n-6: n-3 PUFA ratio may result in altered gene regulation and expression in downstream pathways resulting in altered protein expression and activity that can negatively affect cell membrane composition and fluidity and organ function. Therefore, increasing the EPA and/or DHA to achieve a ratio closer to the desired 1:1-4 would be beneficial for diet.

PUFAs also have both inflammatory and anti-inflammatory characteristics that appear to mediate cellular activities, and the ratio of n-3 to n-6 can alter the impact on cellular metabolism. Generally, n-6 PUFAs are pro-inflammatory, and n-3 are anti-inflammatory, but this is context dependent. DHA is reported to be more potent than EPA for some anti-inflammatory uses.

A continuing need exists for method of selectively increase or enrich production of specific PUFAs, such as EPA, in microalgae such as Thraustochytrids, without decreasing the overall lipid production. In other words, a need exists for increasing the potency of the PUFA, i.e., the percentage of PUFAs relative to the total amount of fatty acids. In addition, there is a need for selective increase in the percentage of a specific PUFA such as EPA without decreasing the percentage of PUFAs.

### BRIEF SUMMARY OF THE INVENTION

The present disclosure provides a method for producing lipids enriched in omega-3 polyunsaturated fatty acids (PUFAs), such as EPA and DHA, and/or selectively enriched in EPA, by adjusting the micro- and/or macronutrients in the fermentation media of the PUFA producing microalgae.

It has been discovered that strains of Thraustochytrids such as Schizochytrium that generate both EPA and DHA can be manipulated by fermentation media refinements to generate varying ratios of EPA (omega-3) to DHA (omega-3). It has also been discovered that fat content and/or titer, and/or PUFA content of either EPA or DHA can be selectively increased.

In one embodiment, the PUFA content and/or ratios can be manipulated by altering the media macronutrients or micronutrients. In some embodiments, altering the macronutrients does not adversely affect the total fatty acid content produced. In another embodiment, altering the macronutrients or micronutrients does not require altering growth phases or rates as has been reported previously to increase lipid production in some species (See Boelen et al., Aquaculture International. 2017; 25:277-87).

In one specific embodiment, total fat content is increased by altering macronutrients, resulting in increased levels of PUFAs via increased lipid (fat) production.

In another specific embodiment, total fat content is not increased, and may be decreased, but the proportion of PUFAs as a percentage of total fat content ((gPUFA/gFat)*100) is increased. In other words, the macronutrient adjustment selectively enriches the lipid profile in favor of PUFA production.

In yet another specific embodiment, higher ratios of EPA to DHA are achieved by altering nutrient content in the fermentation media.

In a first embodiment, EPA content in the oil can be increased through lowering the sodium to potassium (Na:K) ratio.

In a specific embodiment, the ratio is lowered by increasing potassium.

In a further embodiment, the potassium is potassium sulfate, K₂SO₄.

In one embodiment, the increased potassium to sodium ratio results in an increase in the percentage of EPA as a percent of the total fat content.

In another specific embodiment, the Na:K ratio is lowered by decreasing the sodium.

In a specific embodiment, the percentage of eicosapentaenoic acid as a percent of total lipids as a result of decreased sodium to potassium ratio is from about 8 to about 20%, optionally about 9 to about 18%, optionally about 10-16%, optionally about 11-14%, and including about 8%, about 9%, about 10%, about 11%, about 12%, about 13%, about 14%, about 15%, about 16%, about 17%, about 18%, about 19%, and about 20%. The amount of increase is relative to the original amount, e.g., a starting amount of EPA of 20% was increased to an amount of EPA of 30%, which provided an increased amount of 10%.

In other embodiments, the EPA concentration is 1% to 60%, 1% to 50%, 1% to 40%, 1% to 30%, 1% to 25%, or 1% to 20% EPA by weight of the total fatty acids.

In a further embodiment, the Na:K ratio is adjusted from about 1.5 to about 3.5 to increase the percent of EPA production.

In a specific embodiment, the Na:K ratio is about 1.87, about 1.89 or about 3.36.

In another specific embodiment an Na:K ratio is about 2.0 to about 3.0 to increase the percent of EPA production.

In a further embodiment, the decreased Na:K ratio has no adverse effect on the percentage of DHA or fat produced, but selectively increases the percent of EPA production.

In a second embodiment, the nutrient adjustment includes modifying the nitrogen content and/or the phosphorus content.

In one aspect, the nitrogen to phosphorus ratio (N:P) is adjusted to increase the N:P ratio.

In one embodiment, a higher nitrogen to phosphorus ratio selectively increases EPA production.

In a further embodiment, increasing the N:P ratio increases EPA content as a percentage of the total lipids by at least 5%, at least 10%, at least 12%, at least 13%, at least 14%, or at least 15%.

In a specific embodiment the N:P ratio is between 8% to 14% to increase EPA percentage.

In one embodiment, the N:P ratio is increased by decreasing the concentration of phosphorus.

In a specific embodiment, the phosphorus is KH₂PO₄.

In one aspect of this embodiment, decreasing phosphorus is not associated with a decrease in total lipid content.

In another embodiment, the N:P ratio is increased by increasing the concentration of nitrogen.

In a specific embodiment, the N:P ratio that increases EPA percentage is between about 9.5 and about 15, optionally between about 10 and 14.5.

In a specific embodiment, increasing nitrogen content to achieve an N:P ratio of about 9.85 to about 14.26 results in increased EPA, both titer and as a percentage of total fat content.

In a specific embodiment, an N:P ratio is from about 7 to about 13 results in increased total fat content and fat titer.

In one embodiment, the total fat content (percentage) is increased by about 1%, by about 2%, by about 3% by about 4% or by about 5%.

In one embodiment, an N:P ratio of between about 7.5 and about 10, optionally, about 9.85 increases total fat content (percentage) and fat titer (g/kg).

In a specific embodiment, an N:P ratio between about 9.5 and about 14 increases EPA as a percentage of total fat content.

In one embodiment, EPA titer is increased to about 15%.

In another embodiment, an N:P ratio of about 15 to about 16 increases the DHA titer.

In a specific embodiment, an N:P ratio of about 9.85 results in the highest percent fat and increases DHA and EPA titer.

In a specific embodiment, the N:P ratio is adjusted during the production phase.

In another specific embodiment, the N:P ratio is adjusted during the growth phase.

In a third embodiment, altering the source of nitrogen and/or adding nitrogen during the production phase affects the fat and PUFA production.

In one embodiment, nitrogen is added during the production phase to increase PUFA production.

In one embodiment, the nitrogen source used is ammonium sulfate or glutamate, including but not limited to monosodium glutamate.

In another embodiment, the nitrogen source used is ammonia gas and NH₄OH.

In a specific embodiment, increasing glutamate during the production phase increases the production of PUFAs by about 25% or more.

In one embodiment, addition of glutamate during the production phase increases EPA or DHA by at least 5%, at least 10%, at least 15%, at least 20%, at least 25%, at least about 30%, about least about 35%, at least about 40%, or at least about 45%.

In another specific embodiment, addition of glutamate during the production phase increases total lipid titer (g/kg) and consequently increases both EPA and DHA production by at least 50% to up to 100%.

In a specific embodiment, EPA titer is increased from about 0.6 g/kg to 1.2 g/kg.

In a further specific embodiment, addition of glutamate during the production phase increases the q-rate of total fat. As used herein the q-rate is grams of fat produced by grams of cells over t-hours (g-fat/(g-cells*hr).

In a specific embodiment, glutamate fed during the production phase increases the q-rate of EPA over DHA as compared to control fermentations where the DHA is normally about twice that of EPA.

In a specific embodiment, the glutamate is added at about 2.9 g/L (20 mM) during the production phase.

In another embodiment, the nitrogen source is ammonium sulfate.

In one embodiment, addition of ammonium sulfate increases the PUFA production as a percentage of the total fat (potency), although total fat content is decreased compared to addition of monosodium glutamate.

In a further embodiment, the ammonium sulfate is added at 10 mM or 0.36 g/L of ammonium ions).

Reducing the sodium to potassium and/or increasing the nitrogen to phosphorous ratio or altering the source of nitrogen during production can be done in combination with other adjustments to fermentation conditions to increase fat content or selectively increase EPA.

In yet a further embodiment of the invention, EPA and DHA potency, which is the grams of DHA+EPA as a proportion of the total grams of fat can be increased or decreased.

Unless otherwise specified, increases in e.g., EPA, mean increased EPA as a percent of total lipids produced, or increased titer of EPA (e.g., in grams) due to increased lipid production.

In a fourth embodiment, adjusting the carbon source, such as glucose or glycerol in combination with adjusting the macronutrients, also can increase EPA.

In a one embodiment, mixed feeding glucose and glycerol during the production phase can increase both EPA and total lipid titers.

In one embodiment, EPA percentage increased from about 20 to about 25%, more specifically from about 19% to about 23%.

In another embodiment, lipid titer increased by about 3% to about 10%, optionally about 4%, about 5%, about 6%, about 7%, about 8%, about 9% or about 10%.

In a specific embodiment, a glucose concentration of about 5 g/L to about 60 g/L and a glycerol concentration of greater than 1 g/L up to about 30 g/L can be used in the production phase.

In a fifth embodiment, controlling dissolved oxygen can also be used in combination with the nutrient adjustments to e.g., increase EPA production.

In a specific embodiment, dissolved oxygen from about 5-15%, optionally about 10%, in the lipid production phase increases EPA production in combination with micronutrient adjustments. In specific embodiments, the dissolved oxygen is 5%, 6%, 7%, 8%, 9%, 10%, 11%, 12%, 13%, 14% or 15%.

In one embodiment, probes are calibrated at ambient pressure and temperature of 22.5° C. at a dissolved oxygen content of about 9 mg/L.

In another embodiment, temperature adjustments made concomitantly with the nutrient adjustments also can also increase EPA titer and/or decrease the EPA/DHA ratio. In a specific embodiment, the temperature is decreased to about 20° C. during the lipid production phase.

For example, EPA can be increased from about 22% to about 28% by decreasing the temperature to about 20° C. In specific embodiments, EPA is increased by about 22%, 23%, 24%, 25%, 26%, 27% or 28%.

In a further embodiment, pH adjustments also can increase the percentage of EPA. In one embodiment, maintaining a pH range between 7.5-8.0 increases EPA percentage. In some embodiments, the pH of the culture medium is adjusted to greater than 7.5, greater than or equal to about 7.6, preferably greater than or equal to about 7.7, more preferably greater than or equal to about 7.8, such as greater than or equal to about 7.9 or greater than or equal to about 8.0 during the lipid production (fermentation) phase. The pH can be adjusted using, for example, a base such as sodium hydroxide (e.g., IN NaOH), ammonium hydroxide, calcium hydroxide, magnesium hydroxide, or potassium hydroxide.

In a specific embodiment, the pH is maintained at 8.0.

In one embodiment a pH of 8.0 increases the percentage of EPA from about 8.5% to about 15%.

In a sixth embodiment, the present invention is also directed to a microbial oil produced by the methods described herein and to products comprising or made from the oil.

In one embodiment, the microbial oil has a higher percentage of EPA compared to DHA or other PUFAS.

In another embodiment, the microbial oil has a higher percentage of total fat, optionally including both DHA and EPA, compared to oil produced not using the media refinement described herein.

In a specific embodiment, the microbial oil comprises at least about 25%, at least about 30%, about least about 35%, at least about 40%, or at least about 45% by weight EPA.

In a further embodiment, the oil is used in products including but not limited to food products, dietary supplements, pharmaceuticals, and cosmetics.

### BRIEF DESCRIPTION OF THE DRAWINGS

FIGS. 1A-1C show the results of testing different Na:K ratios on the production of EPA in shake flasks. FIG. 1A shows that a ratio of 2.0 to just under 3.0 increased the percentage of EPA (gEPA/gFat*100). FIG. 1B shows that the range of Na:K ratios did not have any adverse effect on DHA production. FIG. 1C shows that the lower Na:K ratios slightly lowered the overall percentage of fat produced.

FIGS. 2A-2C depict results on EPA and DHA production using a range of Na:K ratio in a 10 L fermentation of Schizochytrium. FIG. 2A shows that Na:K ratios of 1.87 and 2.5 resulted in a higher percent of EPA production than a ratio of 3.56. FIG. 2B shows that adjusting the Na:K ratios had no effect on DHA percentage. FIG. 2C shows no adverse effect on the percentage of total fat in the 10 L fermentations.

FIGS. 3A-3E show results on EPA and DHA production by Schizochytrium at differing N:P ratios from N:P 7.74 to 15.95 via a KH₂PO₄ gradient in shake flask experiments. FIG. 3A shows the fat titers and FIG. 3B shows percent fat at day 10. The N:P=9.85 obtained the highest percent fat overall and also high titers. FIG. 3C shows that EPA titers decreased at the N:P 7.74 and 9.85 and were higher at the other ratios. FIG. 3D shows that the percentage of EPA increase at an N:P of 14.26. FIG. 3D shows the effects of the ratios on DHA percentage.

FIGS. 4A-4D show the effect of feeding nitrogen during the production on EPA and DHA production in Schizochytrium. Nitrogen was added as either ammonium sulfate or monosodium glutamate (MSG) at day 5. FIG. 4A shows the impacts on glucose consumption.

FIG. 4B shows that addition of both nitrogen sources, but especially glutamate, increased lean biomass after 10 days. FIG. 4C shows that the percent of fat was decreased by the addition of nitrogen. FIG. 4D shows that MSG increased fat titer compared to the control while ammonium sulfate decreased titer.

FIGS. 5A-5E show the effects of feeding nitrogen on EPA and DHA production. Ammonium sulfate increased the percent of PUFAs relative to that of total lipids (FIG. 5A) despite a decrease in fat titer. FIG. 5B shows that the percentage of EPA and DHA after 10 days with the nitrogen feed was not significantly different from the control. FIGS. 5C and FIG. 5D, show the increase in EPA and DHA titers, respectively, upon addition of glutamate.

FIGS. 6A-6E show the effects on feeding nitrogen on the q-Rates for fat, DHA and EPA.

FIGS. 6A-FIG. 6C show the effect of feeding glutamate on fat, DHA and EPA q-Rates, respectively.

FIG. 6C and FIG. 6D show the effects of feeding ammonium sulfate on DHA and EPA q-Rates.

### DETAILED DESCRIPTION OF THE INVENTION

It has been reported that nitrogen starvation is beneficial for lipid accumulation but will decrease cell growth rate and EPA percentage in total fatty acids. During standard fermentation of Thraustochytrids, DHA increases as a percentage of total fat content but EPA decreases.

Over the course of fermentation, the percentage of EPA drops while concurrently the percentage of DHA in fat increases. The increase in the percentage of DHA is driven by a major increase in DHA titer relative to the increase in EPA titer.

The methods of the present invention relate to increasing the percentage of EPA by manipulating the macronutrients either by increasing the total fat percentage or by selectively increasing EPA percentage and titer in the fat produced.

### Microorganisms

The methods described herein include fermenting and recovering lipids from a population of microorganisms. The population of microorganisms described herein can be algae (e.g., microalgae), fungi (including yeast), bacteria, or protists. Preferably, the microorganism includes Thraustochytrids of the order Thraustochytriales, more specifically. Thraustochytriales of the genus Thraustochytrium and Schizochytrium.

For purposes of the present invention, strains described as Thraustochytrids can include the following organisms: Order: Thraustochytriales; Family: Thraustochytriaceae; Genera: Thraustochytrium (Species: sp., arudimentale, aureum, benthicola, globosum, kinnei, motivum, multirudimentale, pachydermum, proliferum, roseum, striatum), Ulkenia (Species: sp., amoeboidea, kerguelensis, minuta, profunda, radiata, sailens, sarkariana, schizochytrops, visurgensis, yorkensis), Schizochytrium (Species: sp., aggregatum, limnaceum, mangrovei, minuturn, octosporum), Japonochytrium (Species: sp., marinum), Aplanochytrium (Species: sp., haliotidis, kerguelensis, profunda, stocchinoi), Althornia (Species: sp., crouchii), or Elina (Species: sp., marisalba, sinorifica). For the purposes of this invention, species described within Ulkenia will be considered to be members of the genus Thraustochytrium. Aurantiochytrium, Oblongichytrium, Botryochytrium, Parietichytrium, and Sicyoidochytrium are additional genuses encompassed by the present invention.

In a preferred embodiment, the Thraustochytrid of the invention is Schizochytrium, Thraustochytrium, or mixtures thereof. In an embodiment, the Thraustochytrid is from a species selected from Schizochytrium sp., Schizochytrium aggregatum, Schizochytrium limacinum, Schizochytrium minutum, Thraustochytrium sp., Thraustochytrium striatum, Thraustochytrium aureum, Thraustochytrium roseum, Japonochytrium sp., and strains derived therefrom. In a more preferred embodiment, the Thraustochytrid is Schizochytrium.

The population of microorganisms includes Thraustochytriales as described in U.S. Pat. Nos. 5,340,594 and 5,340,742, which are incorporated herein by reference in their entireties. The microorganism can be a Thraustochytrium species, such as the Thraustochytrium species deposited as ATCC Accession No. PTA-6245 (i.e., ONC-T18).

Some embodiments of the invention are further directed to a culture comprising a mutant strain, such as deposited under ATCC Accession No. PTA-9695.

In another embodiment, the microorganisms are described in U.S. Pat. No. 10,798,952, under ATCC Accession No. PTA-10208, PTA-10212, PTA-10213, PTA-10214, PTA-10215, PTA-10208, PTA-10209, PTA-10210, or PTA-10211, or mixtures thereof.

The methods herein also include using genetically modified Thraustochytrids. Such modified microorganisms may for example contain a polyketide synthase (PKS) for producing PUFAs, or otherwise engineered enhance PUFA production. Such modified organisms are described, for example, in U.S. Pat. Nos. 8,309,796; 8,426,686; 8,859,855; 8,940,884; 9,012,616; 9,382,521; 9,133,463; 9,540,666; 9,873,880; 10,085,465, 10,087,430 and 10,973,837.

### Culture and Fermentation

It has been surprisingly discovered that adjusting certain concentrations of micronutrients and macronutrients in the culture medium (media refinement) can selectively increase EPA percentage and/or titer. Under some conditions, the DHA content and/or fat content is not reduced despite the increase in EPA.

The microorganisms, such as Thraustochytrids, described herein can be cultured in large scale industrial bioreactors. Notwithstanding the specific adjustments to the fermentation media disclosed herein, general fermentation conditions are described in e.g., U.S. Pat. No. 9,045,785, incorporated herein by reference. The microorganisms provided herein are cultivated under conditions that increase biomass and/or production of a compound of interest (e.g., oil or total fatty acid content).

The production of desirable lipids can be enhanced by culturing cells according to methods that involve a shift of one or more culture conditions in order to obtain higher quantities of desirable PUFAs.

The present disclosure is also directed to an isolated biomass comprising a fatty acid profile of the disclosure. In some embodiments, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% of the dry cell weight of the biomass are fatty acids. In some embodiments, about 20% to about 55%, about 20% to about 60%, about 20% to about 70%, about 20% to about 80%, about 30% to about 55%, about 30% to about 70%, about 30% to about 80%, about 40% to about 60%, about 40% to about 70%, about 40% to about 80%, about 50% to about 60%, about 50% to about 70%, about 50% to about 80%, about 55% to about 70%, about 55% to about 80%, about 60% to about 70%, or about 60% to about 80% by weight of the dry cell weight of the biomass are fatty acids, and wherein at least about 50% by weight of the fatty acids are omega-3 fatty acids.

In an embodiment, the weight ratio of EPA to DHA is at least about 0.5:1 to 2:1, preferably 0.5:1 to 1:1. In an embodiment, the weight ratio of EPA to DHA is equal to or above 0.5:1, or preferably equal to or above 1:1, i.e., 0.5:1, 0.6:1, 0.7:1, 0.8:1, 0.9:1, 1:1, 1.5:1, 2:1, etc.

Generally, PUFAs are produced by culturing the microorganisms in a two-phase process that comprises increasing the biomass in an initial growth phase with aeration, followed by a low-oxygen phase in which the lipid synthesis occurs (fermentation phase).

Optionally, cells are cultured first under conditions that maximize biomass, followed by a shift of one or more culture conditions to conditions that favor lipid productivity. In addition to the specific adjustments to media macronutrients described herein, conditions that are shifted can include oxygen concentration, C:N ratio, temperature, and combinations thereof. Optionally, a two-stage culture is performed in which a first stage favors biomass production (e.g., using conditions of high oxygen (e.g., generally or relative to the second stage), low C:N ratio, and ambient temperature), followed by a second stage that favors lipid production (e.g., in which oxygen is decreased, C:N ratio is increased, and temperature is decreased).

Thraustochytrids, for example, are typically cultured in saline media. Optionally, Thraustochytrids can be cultured in medium having a salt concentration from about 2.0 g/L to about 50.0 g/L. Optionally, Thraustochytrids are cultured in media having a salt concentration from about 2 g/L to about 35 g/L (e.g., from about 18 g/L to about 35 g/L). Optionally, the Thraustochytrids described herein can be grown in low salt conditions. For example, the Thraustochytrids can be cultured in a medium having a salt concentration from about 5 g/L to about 20 g/L (e.g., from about 5 g/L to about 15 g/L). The culture media optionally include NaCl. Optionally, the media include natural or artificial sea salt and/or artificial seawater.

The chloride concentration in culture media can be reduced (i.e., lower in amount) as compared to traditional methods. The culture media can include non-chloride-containing sodium salts (e.g., sodium sulfate) as a source of sodium. For example, a significant portion of the total sodium can be supplied by non-chloride salts such that less than about 100%, 75%, 50%, or 25% of the total sodium in culture media is supplied by sodium chloride.

Optionally, the culture media have chloride concentrations of less than about 3 g/L, 500 mg/L, 250 mg/L, or 120 mg/L. For example, culture media have chloride concentrations of between and including about 60 mg/L and 120 mg/L. Examples of non-chloride sodium salts suitable for use in accordance with the present methods include, but are not limited to, soda ash (a mixture of sodium carbonate and sodium oxide), sodium carbonate, sodium bicarbonate, sodium sulfate, and mixtures thereof (See, e.g., U.S. Pat. Nos. 5,340,742 and 6,607,900, the entire contents of each of which are incorporated by reference herein).

Media for Thraustochytrid culture can include any of a variety of carbon sources. Examples of carbon sources include fatty acids; lipids; glycerols; triglycerols; carbohydrates such as glucose, starch, celluloses, hemicelluloses, fructose, dextrose, xylose, lactulose, galactose, maltotriose, maltose, lactose, glycogen, gelatin, starch (corn or wheat), acetate, m-inositol (derived from corn steep liquor), galacturonic acid (derived from pectin), L-fucose (derived from galactose), gentiobiose, glucosamine, alpha-D-glucose-1-phosphate (derived from glucose), cellobiose, dextrin, and alpha-cyclodextrin (derived from starch); sucrose (from molasses); polyols such as maltitol, erythritol, adonitol and oleic acids such as glycerol and tween 80; amino sugars such as N-acetyl-D-galactosamine, N-acetyl-D-glucosamine and N-acetyl-beta-D-mannosamine; and any kind of biomass or waste stream.

Media include carbon sources at a concentration of about 5 g/L to about 200 g/L, about 5 g/L to about 100 g/L, about 10 g/L to about 50 g/L, or about 10 g/L to about 30 g/L.

In one embodiment, the carbon source is glucose. In a specific embodiment the glucose is present in a concentration of about 50 g/L. In the mixed feed embodiment using glucose and glycerol, glucose concentration can be about 5 to 60 g/L while the glycerol concentration can be about >1 g/L to about 30 g/L. Media can have a C:N (carbon to nitrogen) ratio between about 1:1 and about 40:1.

In a specific embodiment, the mixed feed includes at least about 15% more carbon than glycerol. In the mixed feed embodiment using glucose and glycerol, glucose concentration can be about 5 to 60 g/L while the glycerol concentration can be about >0 g/L to about 30 g/L. Media can have a C: N (carbon to nitrogen) ratio between about 1:1 and about 40:1.

In a specific embodiment, a mixed feed of glucose and glycerol fed during the production phase can selectively increase EPA in the oil as well as increase DHA and EPA titers.

In a specific embodiment, average EPA levels can increase by at least about 20% due to increased fat production from the mixed feed.

In another specific embodiment, EPA percentage increases from about 19% to about 23% using a mixed feed of glucose and glycerol fed during the production phase.

In a further specific embodiment, EPA titer can increase from about 105 g/kg to about 130 g/kg using mixed feed of glucose and glycerol fed during the production phase.

When two-phase cultures are used, typical media can have a C:N ratio of between and including about 1:1 to about 5:1 for the first phase, then about 1:1 to about 1:0 (i.e., no or minimal nitrogen) in the second phase. As used herein, the term minimal refers to less than about 10% (including all ranges between 0.1% and 10%).

Media for Thraustochytrids culture can include any of a variety of nitrogen sources. Exemplary nitrogen sources include ammonium solutions (e.g., NH₄ in H₂O), ammonium or amine salts (e.g., NH₂SO₄, (NH₄)₃PO₄, NH₄NO₃, NH₄OCH₂CH₃ (ammonium acetate)), peptone, tryptone, yeast extract, malt extract, fish meal, sodium glutamate, soy extract, casamino acids and distiller grains.

A typical media for growth of Thraustochytrids is shown below in Table 1, reproduced from Table 2 of U.S. Pat. No. 9,045,785:

TABLE 1

| Ingredient | concentration | | ranges |
|---|---|---|---|
| Na₂SO₄ | g/L | 8.8 | 0-25, 2-20, or 3-10 |
| NaCl | g/L | 0.625 | 0-25, 0.1-10, or 0.5-5 |
| KCl | g/L | 1.0 | 0-5, 0.25-3, or 0.5-2 |
| MgSO₄•7H₂O | g/L | 5.0 | 0-10, 2-8, or 3-6 |
| (NH₄)₂SO₄ | g/L | 0.42 | 0-10, 0.25-5, or 0.05-3 |
| CaCl₂ | g/L | 0.29 | 0.1-5, 0.15-3, or 0.2-1 |
| T 154 (yeast extract) | g/L | 1.0 | 0-20, 0.1-10, or 0.5-5 |
| KH₂PO₄ | g/L | 1.765 | 0.1-10, 0.5-5, or 1-3 |

Post autoclave (Metals)

| | | | |
|---|---|---|---|
| Citric acid | mg/L | 46.82 | 0.1-5000, 10-3000, or 40-2500 |
| FeSO₄•7H₂O | mg/L | 10.30 | 0.1-100, 1-50, or 5-25 |
| MnCl₂•4H₂O | mg/L | 3.10 | 0.1-100, 1-50, or 2-25 |
| ZnSO₄•7H₂O | mg/L | 9.3 | 0.01-100, 1-50, or 2-25 |
| CoCl₂•6H₂O | mg/L | 0.04 | 0-1, 0.001-0.1, or 0.01-0.1 |
| Na₂MoO₄•2H₂O | mg/L | 0.04 | 0.001-1, 0.005-0.5, or 0.01-0.1 |
| CuSO₄•5H₂O | mg/L | 2.07 | 0.1-100, 0.5-50, or 1-25 |
| NiSO₄•6H₂O | mg/L | 2.07 | 0.1-100, 0.5-50, or 1-25 |

Post autoclave (Vitamins)

| | | | |
|---|---|---|---|
| Thiamine | mg/L | 9.75 | 0.1-100, 1-50, or 5-25 |
| Ca½-pantothenate | mg/L | 3.33 | 0.1-100, 0.1-50, or 1-10 |
| Biotin | mg/L | 3.58 | 0.1-100, 0.1-50, or 1-10 |

Post autoclave (Carbon)

| | | | |
|---|---|---|---|
| Glucose | g/L | 30.0 | 5-150, 10-100, or 20-50 |

Nitrogen Feed:

| | | | |
|---|---|---|---|
| NH₄OH | mL/L | 23.6 | 0-150, 10-100, 15-50 |

The carbon source of glucose, glycerol or both is typically in the range of 30 to 50 g/L. In the growth phase, nitrogen is added at a concentration of about 23.6 ml/L but can range from about 5 g/L to about 30 g/L including all ranges in between.

Typical cultivation conditions including using a pH of about 6.5 to about 9.5, about 6.5 to about 8.0, or about 6.8 to about 7.8; a temperature of about 15° C. to about 30° C.; about 18° C. to about 28° C.; or about 21° C. to about 23° C.; dissolved oxygen of about 0.1 to about 100% saturation; about 5 to about 50% saturation; or about 10 to about 30% saturation.

The present invention demonstrates that total fat content and/or titer is increased by altering macronutrients, or timing of their addition, resulting in increased levels or titers of PUFAS via increased lipid (fat) production, or selective enrichment of specific PUFAs.

Alternatively, total fat content is not increased, and may be decreased, but the selective proportion of PUFAs as a percentage of total fat content is increased.

The examples below demonstrate that total fat and or the percentage and/or titer of EPA content in the oil can be increased through i) lowering the sodium to potassium (Na:K) ratio, optionally, by increasing potassium levels in the media; ii) lowering the nitrogen to phosphorus (N:P) ratio, optionally, by adjusting the concentration of phosphorus; and iii) feeding nitrogen in the production phase, optionally, glutamate, or combinations thereof.

For example, it was found that a sodium to potassium ratio (Na:K) of from about 1:75 to about 3.75 during fermentation resulted in increased percentage of EPA without negatively impacting the percentage of total fat or DHA under standard fermentation conditions. A Na:K ratio of between about 2 and about 3 was even more optimal at increasing EPA. The Na:K ratio was achieved by increasing the amount of K₂SO₄ and resulted in the increase in the percentage of EPA of from about 8.5% to about 12%.

It was further found that an N:P ratio of between about 9 and about 12, optionally about 9.85, fed during the production phase increased total fat (both titer and percentage).

It was further unexpectedly found that certain N:P ratios (about 9 to about 15) selectively increased EPA percentage by at least 10%, at least 12%, at least 13%, at least 14%, or at least 15%. The N:P ratio was decreased by increasing the concentration of KH₂PO₄.

Further, it was found that the DHA percentage increased at day 7 with a lower N:P ratio.

The present invention also provides a method of adding nitrogen during the production phase to affect the fat and PUFA production. The nitrogen source was also determined to affect the lipid production during this step.

In one embodiment, a nitrogen source added during the lipid production phase is ammonium sulfate, NH₄SO₄ or glutamate, including but not limited, to monosodium glutamate.

It was unexpectedly found that increasing glutamate increases the production of PUFAs by about 20%, about 21%, about 22%, about 23%, about 24%, or about 25%, or more.

In another specific embodiment, addition of glutamate increases total lipid titer (g/kg) and consequently increases both EPA and DHA titer, compared to controls.

In a further specific embodiment, addition of glutamate during the production phase increases the q-rate total fat. As used herein the q-rate is grams of fat produced by grams of cells over t-hours (g-fat/(g-cells*hr).

In a specific embodiment, glutamate fed during the production phase increases the q-rate of EPA over DHA as compared to control fermentations where the DHA is normally about twice that of EPA.

In a specific embodiment, the glutamate is added at from about 2.5 g/L to about 3.5 g/L, optionally about 2.9 g/L (20 mM) during the production phase.

In another embodiment, the nitrogen source is ammonium sulfate.

In one embodiment, addition of ammonium sulfate increases the PUFA production as a percentage of the total fat (potency), although total fat content is decreased compared to addition of monosodium glutamate.

In a further embodiment, the ammonium sulfate is added at about 10 mM or about 0.36 g/L of ammonium ions.

Reducing the sodium to potassium and/or N:P concentrations can be done in combination with other adjustments to fermentation conditions to increase fat content or selectively increase EPA.

The pH of the medium can be adjusted to between and including 3.0 and 10.0 using acid or base, where appropriate, and/or using the nitrogen source. Optionally, the medium is adjusted to a pH from 7.5-8.0 which is shown to increase EPA. The medium can be sterilized.

In some embodiments, the fermentation volume (volume of culture) is at least about 2 liters, at least about 10 liters, at least about 50 liters, at least about 100 liters, at least about 200 liters, at least about 500 liters, at least about 1000 liters, at least about 10,000 liters, at least about 20,000 liters, at least about 50,000 liters, at least about 100,000 liters, at least about 150,000 liters, at least about 200,000 liters, or at least about 250,000 liters. In some embodiments, the fermentation volume is about 2 liters to about 300,000 liters, about 2 liters, about 10 liters, about 50 liters, about 100 liters, about 200 liters, about 500 liters, about 1 000 liters, about 10,000 liters, about 20,000 liters, about 50,000 liters, about 100,000 liters, about 150,000 liters, about 200,000 liters, about 250,000 liters, or about 300,000 liters.

Cells can be cultivated for anywhere from 1 day to 60 days. Optionally, cultivation is carried out for 14 days or less, 13 days or less, 12 days or less, 11 days or less, 10 days or less, 9 days or less, 8 days or less, 7 days or less, 6 days or less, 5 days or less, 4 days or less, 3 days or less, 2 days or less, or 1 day or less. Cultivation is optionally carried out at temperatures from about 4° C. to about 30° C., e.g., from about 18° C. to about 28° C.

In one embodiment, a culture temperature for Thraustochytrids of about 20° C. has been shown to significantly increase the percent EPA.

Cultivation can include aeration-shaking culture, shaking culture, stationary culture, batch culture, semi-continuous culture, continuous culture, rolling batch culture, wave culture, or the like. Cultivation can be performed using a conventional agitation-fermenter, a bubble column fermenter (batch or continuous cultures), a wave fermenter, etc.

Cultures can be aerated by one or more of a variety of methods, including shaking. Optionally, shaking ranges from about 100 rpm to about 1000 rpm, e.g., from about 350 rpm to about 600 rpm or from about 100 to about 450 rpm. Optionally, the cultures are aerated using different shaking speeds during biomass-producing phases and during lipid-producing phases. Alternatively, or additionally, shaking speeds can vary depending on the type of culture vessel (e.g., shape or size of flask).

Typically, the level of dissolved oxygen (DO) is higher during the biomass production phase (50 to 100%) than it is during the lipid production phase (about 10-20%) when nitrogen is also exhausted. Thus, DO levels are reduced during the lipid production phase (i.e., the DO levels are less than the amount of dissolved oxygen in biomass production phase). However, lowering the dissolved oxygen during the lipid production phase unexpectedly results in increased EPA.

In one embodiment, DO at about 10% during fat production increase the percentage of EPA from about 23% to about 27%.

### Pasteurization

Optionally, the resulting biomass is pasteurized to kill the cells and inactivate undesirable substances present in the biomass. For example, the biomass can be pasteurized to inactivate compound degrading substances. The biomass can be present in the fermentation media or isolated from the fermentation media for the pasteurization step. The pasteurization step can be performed by heating the biomass and/or fermentation media to an elevated temperature. For example, the biomass and/or fermentation media can be heated to a temperature from about and including 50° C. to about and including 50° C. (e.g., from about and including 50° C. to about and including 90° C. or from about and including 65° C. to about and including 80° C.). Optionally, the biomass and/or fermentation media can be heated from about and including 30 minutes to about and including 120 minutes (e.g., from about and including 45 minutes to about and including 90 minutes, or from about and including 55 minutes to about and including 75 minutes). The pasteurization can be performed using a suitable heating means as known to those of skill in the art, such as by direct steam injection.

### Harvesting and Washing

Optionally, the biomass can be harvested according to methods known to those of skill in the art. For example, the biomass can optionally be collected from the fermentation media using various conventional methods, such as centrifugation (e.g., solid-ejecting centrifuges) or filtration (e.g., cross-flow filtration) and can also include the use of a precipitation agent for the accelerated collection of cellular biomass (e.g., sodium phosphate or calcium chloride).

Optionally, the biomass is washed with water. Optionally, the biomass can be concentrated up to about and including 20% solids. For example, the biomass can be concentrated to about and including 5% to about and including 20% solids, from about and including 7.5% to about and including 15% solids, or from about and including 15% solids to about and including 20% solids, or any percentage within the recited ranges. Optionally, the biomass can be concentrated to about 20% solids or less.

### Hydrolysis

Cell hydrolysis (i.e., cell disruption) can be performed using chemical, enzymatic, and/or mechanical methods. Chemical methods for hydrolyzing the cells can include adding acid to the cells, which is referred to herein as acid hydrolysis. In the acid hydrolysis method, the biomass can be washed with water using, for example, centrifugation, and concentrated as described above prior to hydrolyzing the cells. Optionally, the biomass is concentrated to about 15% solids with water.

Acid is then added to the washed, wet biomass. Optionally, the biomass is not dried prior to adding the acid. Suitable acids for use in the acid hydrolysis step include sulfuric acid, hydrochloric acid, phosphoric acid, hydrobromic acid, nitric acid, perchloric acid, and other strong acids as known to those of skill in the art. A suitable amount of acid can be added to the washed, wet biomass to achieve a final concentration of from about and including 100 mM to about and including 200 mM (e.g., from about and including 120 mM to about and including 180 mM or from about and including 140 mM to about and including 160 mM). Sulfuric acid can be added to the washed, wet biomass to a final concentration of 160 mM.

The resulting mixture including water, biomass, and acid can then be incubated for a period of time to hydrolyze the cells. Optionally, the mixture can be incubated at a temperature of from about and including 30° C. to about and including 200° C. For example, the mixture can be incubated at a temperature of from about and including 45° C. to about and including 180° C., from about and including 60° C. to about and including 150° C., or from about and including 80° C. to about and including 130° C. Optionally, the mixture is incubated in an autoclave at a temperature of 121° C. The mixture can be incubated for a period of time suitable to hydrolyze at least 50% of the cells (e.g., at least 60% of the cells, at least 70% of the cells, at least 80% of the cells, at least 90% of the cells, at least 95% of the cells, or 100% of the cells). The period of time for incubating the cells depends on the incubation temperature. Incubating the mixture at a higher temperature can result in the hydrolysis proceeding at a faster rate (i.e., requiring a shorter period of time for hydrolysis). In some examples, the cells can be incubated at 60° C. for 1 hour. Optionally, the incubation step is performed using direct or indirect pasteurization equipment, such as, for example, a continuous flow thermal system commercially available from MicroThermics (e.g., MicroThermics UHT/HTST Lab 25 EHV Hybrid) (Raleigh, NC).

As described above, cell hydrolysis (i.e., cell disruption) can be performed using enzymatic methods. Specifically, the population of microorganisms can be contacted with one or more enzymes under conditions that cause disruption of the microorganisms. Optionally, the enzyme is a protease. An example of a suitable protease is ALCALASE 2.4L FG (Novozymes; Franklinton, N.C.). Optionally, the cells are not washed with water prior to the enzymatic hydrolysis.

The population of microorganisms can be fermented to float in aqueous media. The fermentation media can be gravity settled in the fermenter and the media can be decanted or otherwise removed to provide the desired concentration of the population of microorganisms. Alternatively, the fermentation media can be concentrated by centrifugation to provide the desired concentration of the population of microorganisms. The population of microorganisms can be concentrated to up to and including 20% solids. For example, the population of microorganisms can be concentrated from about and including 5% to about and including 20% solids, from about and including 7.5% to about and including 15% solids, or from about and including 15% solids to about and including 20% solids, or any percentage within the recited ranges. The population of microorganisms can be concentrated prior to contacting the microorganisms with the one or more enzymes.

The microorganisms can be contacted with the one or more enzymes while the population of microorganisms is in the fermentation medium (i.e., the contacting step occurs in the fermentation medium). Optionally, the enzyme added to the fermentation medium is at a concentration of from about 0.2% to about 0.4% volume/volume (v/v). For example, the enzyme added to the fermentation medium can be at a concentration of from 0.2% (v/v) 0.25% (v/v), 0.30% (v/v), 0.35% (v/v), or 0.4% (v/v).

The contacting step can be performed at a temperature of 70° C. or below. For example, the contacting step can be performed from about and including 1 hour to about and including 20 hours, e.g., from 2 hours to 18 hours, from 4 hours to 16 hours, from 6 hours to 14 hours, or from 8 hours to 12 hours, or any time frame within the recited ranges. Optionally, the contacting step can be performed for about four hours and the hydrolysis temperature can optionally be about 70° C.

Optimum temperature, time, pH, and enzyme concentration depend on the specific enzyme, and a person of ordinary skill in the art would be able to modify the temperature, time, pH, and enzyme concentration as appropriate for a given enzyme.

Optionally, the contacting step is performed in the presence of either about 0.2% or about 0.4% enzyme for about 18 to 20 hours at about 55° C. For example, the contacting step can be performed in the presence of 0.4% enzyme for eighteen hours at 55° C. Alternatively, the contacting step is performed in the presence of 0.4% enzyme for four to six hours at 70° C. Optionally, the contacting step is performed in the absence of surfactants (i.e., no surfactant is present).

Optionally, the cell disruption can be performed using other chemical and mechanical methods as known to those of skill in the art. For example, cell disruption can be performed using alkaline hydrolysis, bead milling, sonication, detergent hydrolysis, solvent extraction, rapid decompression (i.e., the cell bomb method), or high-shear mechanical methods, contact with a chemical, homogenization, ultrasound, milling, shear forces. French press, cold-pressing, heating, drying, osmotic shock, pressure oscillation, expression of an autolysis gene, or combinations of these. Optionally, the cell disruption can be performed using a combination of two or more of the chemical, enzymatic, and/or mechanical methods described herein (e.g., enzymatic hydrolysis in combination with bead-milling). The cell disruption methods can be performed sequentially (e.g., bead-milling followed by enzymatic hydrolysis).

### Extraction

Lipids are extracted from the population of microorganisms in the presence of reduced amounts of organic solvent (i.e., organic solvent extraction) or in the absence of organic solvent.

Optionally, the extraction step is performed using reduced amounts of organic solvent as compared to the amounts of organic solvents needed to extract lipids from whole dry microbial cells. As used herein, the term reduced amounts of organic solvent compared to the amounts of organic solvent needed to extract lipids from whole dry microbial cells means an amount of organic solvent less than that needed to extract lipids from whole dry microbial cells. For example, the ratio of microorganisms or biomass to organic solvent needed for whole dry microbial cells is typically 1:4 or greater. Thus, the reduced amount of organic solvent can provide a ratio of microorganisms or biomass to organic solvent of less than about 1:4. For example, the ratio of microorganisms or biomass to organic solvent for extracting oil from the hydrolyzed wet biomass described herein can be from about and including 1:0.2 to about and including 1:1 (e.g., 1:0.2, 1:0.3, 1:0.4, 1:0.5, 1:0.6, 1:0.7, 1:0.8, or 1:0.9). Optionally, additional amounts of organic solvent can be used, such as up to about a 1:6 ratio of microorganisms or biomass to organic solvent.

Suitable organic solvents for use in the extraction step include hexane, isopropyl alcohol, methylene chloride, dodecane, methanol, ethylated oil, and supercritical carbon dioxide. Polar lipids (e.g., phospholipids) are generally extracted with polar solvents (e.g., chloroform/methanol) and neutral lipids (e.g., triacylglycerols) are generally extracted with nonpolar solvents (e.g., hexane). A preferred solvent is pure hexane.

The organic solvent and microorganisms or biomass can be mixed for a period of time suitable to extract lipids from the microorganisms or biomass. For example, the organic solvent and microorganisms or biomass can be mixed for about 10 minutes or more, 20 minutes or more, 30 minutes or more, 40 minutes or more, 50 minutes or more, 1 hour or more, or 2 hours or more. Subsequently, the lipid can be separated from the remaining components of the mixture by centrifuging the solution.

Optionally, at least about 50% of the lipids theoretically produced by the microorganisms are extracted from the population of microorganisms using this method (i.e., the method provides at least a 50% yield). For example, the yields of lipids extracted from the population of microorganisms can be at least 600%, at least 70%, at least 80%, or at least 90%.

Lipids can also be extracted from the population of microorganisms in the absence of organic solvent. As used herein, in the absence of organic solvent means less than about 0.5% organic solvent based on the weight of the population of microorganisms (e.g., less than about 0.4%, less than about 0.3%, less than about 0.2%, less than about 0.1%, less than about 0.05%, less than about 0.01%, less than about 0.005%, or 0%).

Optionally, the lipids can be extracted from the disrupted microorganisms by using oil (e.g., coconut oil) or biofuel.

Optionally, the oil added during the extraction step can be a nutritional oil (e.g., an oil derived or obtained from a nutritional source). Examples of suitable nutritional oils for use in the methods described herein include coconut oil, palm oil, canola oil, sunflower oil, soy oil, corn oil, olive oil, safflower oil, palm kernel oil, cottonseed oil, and combinations thereof. Derivatives of any of those oils, such as alkylated derivatives (e.g., methylated or ethylated oils), also could be used.

As used herein, biofuel refers to any fuel, fuel additive, aromatic, and/or aliphatic compound derived from a biomass starting material. For example, suitable biofuels for use in the methods described herein can be derived from plant sources or algal sources. Examples of suitable sources for biofuel include algae, corn, switchgrass, sugarcane, sugarbeet, rapeseed, soybeans, and the like.

Optionally, biofuels can be obtained by harvesting oils from a biological source and converting the oils into biofuel. Methods of converting oils obtained from biological sources (e.g., oils obtained from plant and/or algal sources) are known to those of skill in the art. Optionally, the methods of obtaining biofuels can include cultivating an oil-producing biomass (e.g., algae), extracting the oil (e.g., algal oil), and converting the oil (e.g., algal oil) to form a biofuel. Optionally, the oil can be converted to a biofuel using transesterification. As used herein, transesterification refers to a process of exchanging an alkoxy group of an ester by another alcohol. For example, a transesterification process for use in the methods described herein can include converting algal oil, e.g., triglycerides, to biodiesel, e.g., fatty acid alkyl esters, and glycerol. Transesterification can be accomplished by using traditional chemical processes such as acid or base catalyzed reactions, or by using enzyme-catalyzed reactions.

Optionally, at least 40% of the lipids theoretically produced by the microorganisms are extracted from the population of microorganisms using this method (i.e., the method provides at least about a 40% yield). For example, the yields of lipids extracted from the population of microorganisms can be at least about 50%, at least 60%, at least 70%, or at least 80%.

Alternatively, the lipids can be extracted using mechanical methods. The hydrolyzed biomass and microorganisms can be centrifuged, and the lipids can be separated from the remainder of the components. Optionally, the lipids are contained in the upper layer of the centrifuged material and can be removed by suction or decanting, for example, from the other material.

In another alternative, lipid extraction can be achieved without using organic solvents by adjusting the pH, such as lowering the pH or raising the pH to 8 or above to demulsify the lysed cell composition with or without the addition of salt.

Processes of the present invention provide an average lipid production rate of at least about 3 g/kg, at least about 3.5 g/kg, at least about 4 g/kg, or at least about 5 g/kg. Moreover, lipids produced by processes of the invention contain polyunsaturated lipids in the amount greater than about 15%, preferably greater than about 20%, more preferably greater than about 25%, greater than about 30%, greater than about 35%, greater than about 45%, greater than about 50%. Generally, at least about 20% of the lipids produced by the microorganisms in the processes of the present invention are omega-3 and/or omega-6 PUFAs, preferably at least about 30% of the lipids are omega-3 and/or omega-6 PUFAs, more preferably at least about 40% of the lipids are omega-3 and/or omega-6 PUFAs, and most preferably at least about 50% of the lipids are omega-3 and/or omega-6 PUFAs. Alternatively, processes of the present invention provide an average EPA content of at least about 5%, at least about 10%, at least about 15% or at least about 20%.

### Products

Polyunsaturated fatty acids (PUFAs) and other lipids produced according to the method described herein can be utilized in any of a variety of applications, for example, exploiting their biological or nutritional properties. Optionally, the compounds can be used in pharmaceuticals, food supplements, animal feed additives, cosmetics, and the like. Lipids produced according to the methods described herein can also be used as intermediates in the production of other compounds.

Optionally, the lipids produced according to the methods described herein can be incorporated into a final product (e.g., a food or feed supplement, an infant formula, a pharmaceutical, a fuel, etc.). Suitable food or feed supplements for incorporating the lipids described herein into include beverages such as milk, water, sports drinks, energy drinks, teas, and juices; confections such as jellies and biscuits; fat-containing foods and beverages such as dairy products; processed food products such as soft rice (or porridge); infant formulae; breakfast cereals; or the like.

Optionally, one or more produced lipids can be incorporated into a dietary supplement, such as, for example, a multivitamin. Optionally, a lipid produced according to the method described herein can be included in a dietary supplement and optionally can be directly incorporated into a component of food or feed (e.g., a food supplement).

Examples of feedstuffs into which lipids produced by the methods described herein can be incorporated include pet foods such as cat foods; dog foods and the like; feeds for aquarium fish, cultured fish or crustaceans, etc.; feed for farm-raised animals (including livestock and fish or crustaceans raised in aquaculture). Food or feed material into which the lipids produced according to the methods described herein can be incorporated is preferably palatable to the organism which is the intended recipient. This food or feed material can have any physical properties currently known for a food material (e.g., solid, liquid, soft).

Optionally, one or more of the produced compounds (e.g., PUFA) can be incorporated into a pharmaceutical. Examples of such pharmaceuticals include various types of tablets, capsules, drinkable agents, etc. Optionally, the pharmaceutical is suitable for topical application. Dosage forms can include, for example, capsules, oils, granula, granula subtilae, pulveres, tabellae, pilulae, trochisci, or the like.

The lipids produced according to the methods described herein can be incorporated into products or compositions as described herein by combinations with any of a variety of agents. For instance, such compounds can be combined with one or more binders or fillers. In some embodiments, products can include one or more chelating agents, pigments, salts, surfactants, moisturizers, viscosity modifiers, thickeners, emollients, fragrances, preservatives, etc., and combinations thereof. The excipients used can be “pharmaceutically acceptable.” The term “pharmaceutically acceptable” means approved by a regulatory agency of the Federal or a state government or listed in the U.S. Pharmacopeia or other generally recognized international pharmacopeia for use in animals, and more particularly, in humans. Excipients in addition to those discussed herein can include excipients listed in, though not limited to, Remington: The Science and Practice of Pharmacy, 21st ed. (2005). Inclusion of an excipient in a particular classification herein (e.g., “solvent”) is intended to illustrate rather than limit the role of the excipient. A particular excipient can fall within multiple classifications.

The examples below are intended to further illustrate certain aspects of the methods and compositions described herein and are not intended to limit the scope of the claims.

### EXAMPLES

The examples below are intended to further illustrate certain aspects of the methods and compositions described herein and is not intended to limit the scope of the claims.

### Example 1

In this example, experiments were performed to determine the kinetics of growth and fat production of Thraustochytrids when grown in a K₂SO₄ gradient, such that the Na:K ratio was assessed at 59.7; 12.0; 6.0; 3.0; and 2.0. In an initial shake flask experiment, a new cryovial of Schizochytrium was initially thawed and was subsequently grown and transferred several times in media under 10% CO₂ conditions at 22.5° C. A seven-day old stock culture growing in media (below) was used that was inoculated (at 4%: 2 mL inoculum into 50 mL of media) to prepare three inoculum flasks containing media, which were maintained under 10% CO₂ conditions at 22.5° C.

### Pre-Autoclave Components

| Chemical | Concentration (g/L) |
|---|---|
| MgSO₄ 7H₂O | 5.0 |
| KCl | 1.0 |
| K₂SO₄ | GRADIENT |
| (NH₄)2SO₄ | 0.1 |
| CaCl₂ 2H₂O | 0.29 |
| NaCl | 0.625 |
| Na₂SO₄ | 7.52 |
| MSG | 1.0 |
| Yeast extract | 1.0 |
| HEPES | 23.8 |

Post-Autoclave Components Added

| Chemical | Concentration (g/L) |
|---|---|
| KH₂PO₄ | 0.05 |
| Citric Acid | 0.0035 |
| FeSO₄ 7H₂O | 0.0103 |
| Na₂MoO₄ 2H₂O | 0.00004 |
| MnCl₂ 4 H₂O | 0.0031 |
| ZnSO₄ 7 H₂O | 0.0031 |
| CuS O₄ 5 H₂O | 0.00207 |
| NiSO₄ 6 H₂O | 0.00207 |
| Thiamine | 0.0117 |
| CaPanthothenate | 0.0040 |
| Vitamin B12 | 0.000768 |
| Biotin | 0.00434 |
| Glucose | 50 |

After four days of growth, the three inoculum flasks were combined into one flask and used to inoculate the experimental flasks. For each concentration of K₂SO₄ to be tested, eight shake flasks were inoculated at a theoretical dry weight of 0.1 g/L using optical density measurements at 600 nm. Experimental shake flask cultures were grown under 10% CO₂ conditions at 22.5° C. on orbital shakers rotating at 200 rpm with 50 g/L glucose. Each day between day three and day ten of growth, one experimental shake flask culture from each concentration of K₂SO₄ being tested was harvested to determine dry weight. Freeze-dried biomass was analyzed by FAME assay to determine fatty acid profile.

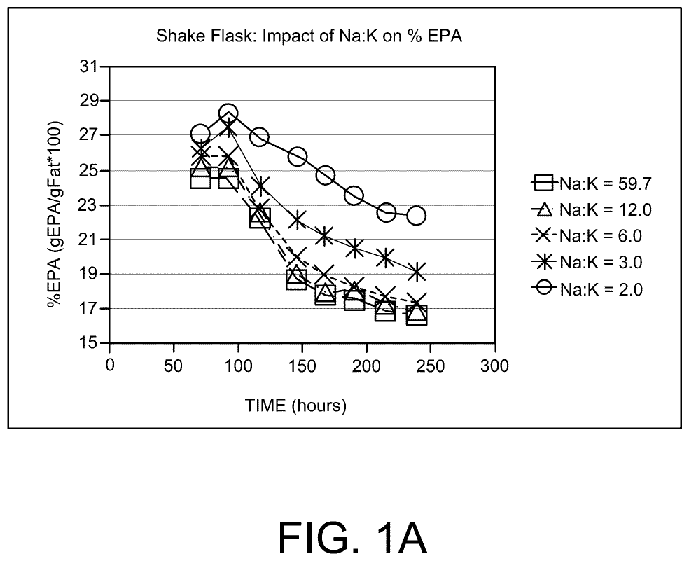

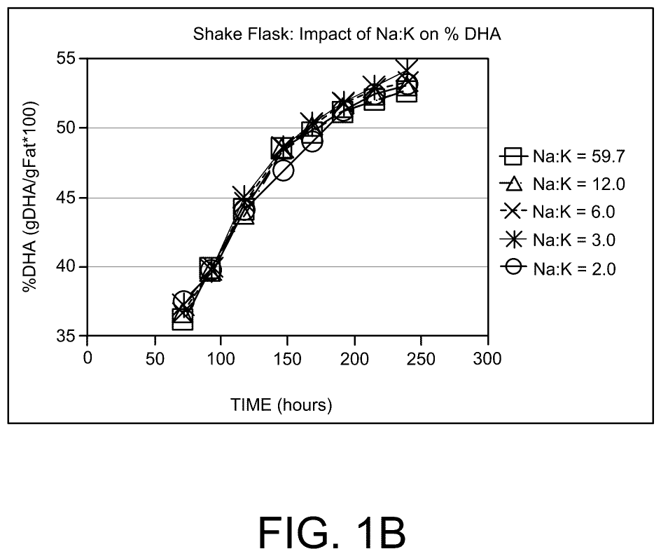

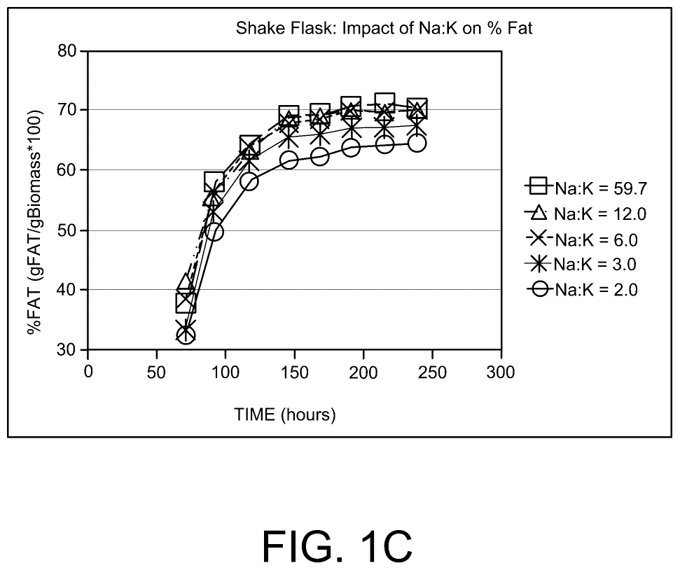

The results are shown in FIGS. 1A-C. A low Na:K ratio of between about 2.0 to about 3.0 increased the percentage of EPA (gEPA/gFat*100) compared to the higher ratios (FIG. 1A).

A low Na:K ratio achieved by increasing the K₂SO₄ can therefore selectively increase the percentage of EPA.

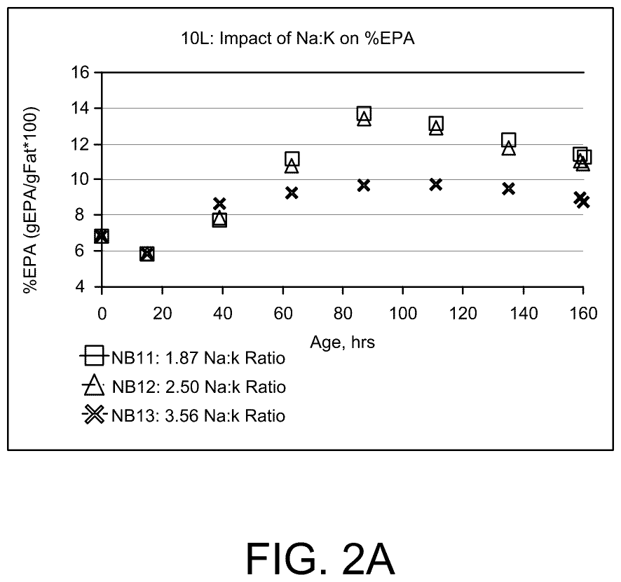

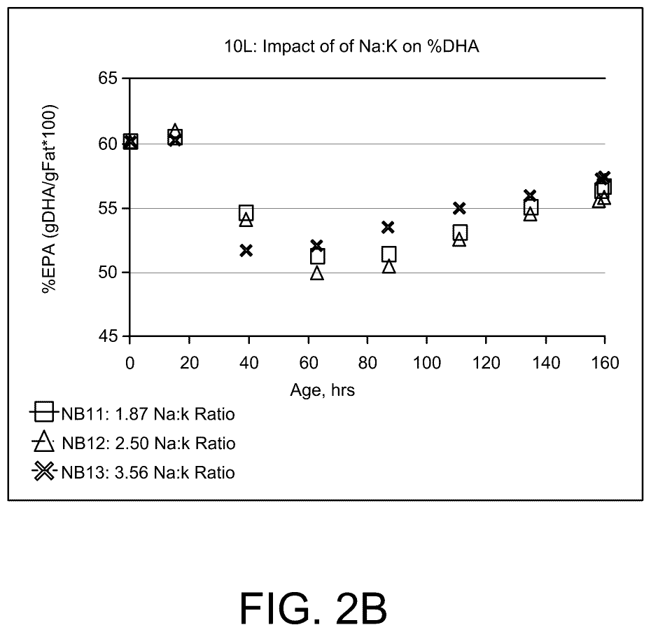

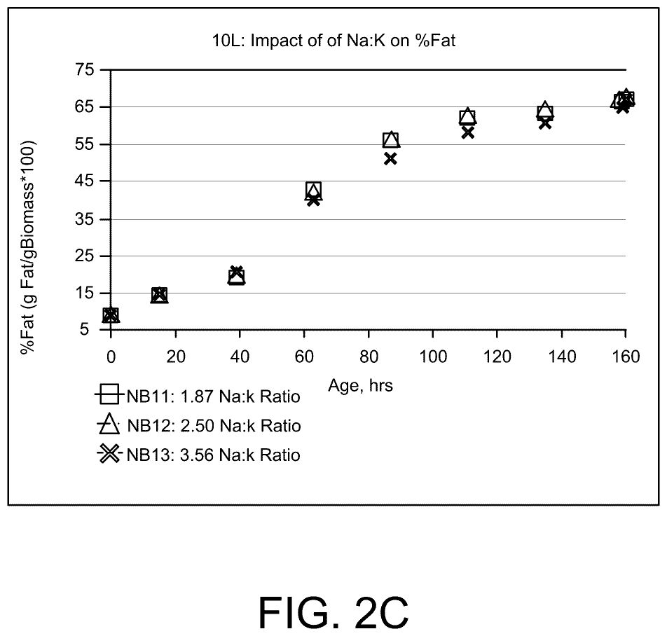

These results were confirmed using a 10 L fermentation under similar conditions and standard dissolved oxygen levels and pH. Specifically, the media used is found in Table 2 of U.S. Pat. No. 9,045,785 (infra). Three Na:K ratios were evaluated in the 10 L fermentations of three strains of Schizochytrium: 1.87; 2.50 and 3.56 over 168 hours. The results are shown in FIGS. 2A-2C. Again, higher amounts of potassium (K₂SO₄) (a lower Na:K ratio) increased the percentage of EPA compared to the lowest level (FIG. 2A). The increased potassium levels had no adverse effect on the DHA percentage or the total fat content (FIGS. 2B and 2C, respectively).

### Example 2

This shake flask experiment investigated the effect altering the nitrogen to phosphorus ratio via a KH₂PO₄ gradient to find an optimal ratio. The media used that set forth in Example 1 with a strain of Schizochytrium. Shake flasks were placed in shakers at 200 rpm at a 24° C. and 5% CO₂ incubator. The experiment was done in triplicate.

The initial concentrations of nutrients in the media are set forth in Table 2, below:

TABLE 2
Initial Concentrations of Nutrients in Media

| | Glucose | Glutamate | PO₄ | NH₄ |
|---|---|---|---|---|
| Condition | (g/L) | (g/L) | (mg/L) | (g/L) |
| 125 mg/L (7.74 N:P) | 44.30 | 1.22 | 176.00 | 0.07 |
| 73 mg/L (9.85 N:P) | 43.76 | 1.24 | 140.00 | 0.07 |
| 30 mg/L (12.38 N:P) | 43.82 | 1.23 | 92.00 | 0.07 |
| 10 mg/L (14.26 N:P) | 42.88 | 1.20 | 75.00 | 0.07 |
| 0 mg/L (15.95 N:P) | 44.03 | 1.24 | 66.00 | 0.07 |

Table 3 below shows the experimental set up of the experiments:

TABLE 3
Experimental set up of flask experiments

| Total N g/L | Total P g/L | | |
|---|---|---|---|
| (NH₄)₂SO₄ + MSG + Angel | KH₂PO₄ + | batched | |
| yeast* | Angel yeast. | KH₂PO₄ g/L | N:P |
| 0.428 | 0.055 | 0.125 | 7.74 |
| | 0.043 | 0.073 | 9.85 |
| | 0.035 | 0.034 | 12.38 |
| | 0.030 | 0.140 | 14.26 |
| | 0.027 | 0.000 | 15.95 |

*Angel yeast contains 11.8 w % nitrogen and 13407 ppm phosphorous

The results are shown in Table 4, which presents results at day 10 of the experiment. The results show higher % fat with lower N:P ratios (NP=7.74 & NP=9.85), however the highest KH₂PO₄ concentration (N:P=7.74), also had the highest amount of saturates (16:0) and lower EPA titer.

TABLE 4
FAME, LFBM and titer results at day 10 (LH240).

| | | | | | DHA + | | | |
|---|---|---|---|---|---|---|---|---|
| | 16:0 | EPA | DHA | Fat | EPA | LFBM | Fat titer | DW |
| KH₂PO₄ | (%) | (%) | (%) | (%) | (g/kg) | (g/kg) | (g/kg) | (g/kg) |
| 125 mg/L (7.74 | 29.9 | 7.65 | 52.95 | 63.3 | 5.79 | 5.81 | 9.98 | 15.78 |
| N:P) | | | | | | | | |
| 73 mg/L (9.85 | 26 | 10.97 | 52.87 | 64.37 | 5.92 | 5.36 | 9.69 | 15.05 |
| N:P) | | | | | | | | |
| 30 mg/L (12.38 | 24.2 | 11.77 | 53.13 | 61.1 | 5.46 | 5.58 | 8.78 | 14.36 |
| N:P) | | | | | | | | |
| 10 mg/L (14.26 | 22.8 | 13.93 | 51.93 | 60.4 | 4.89 | 5.90 | 7.76 | 12.85 |
| N:P) | | | | | | | | |
| 0 mg/L (15.95 | 24.0 | 9.8 | 55.3 | 60 | 4.99 | 5.34 | 8 | 13.34 |
| N:P) | | | | | | | | |

The N:P of 9.85 showed best overall performance in percentage of total fat (64.37±0.42), and DHA+EPA titer (5.92±2.1). The percentage of DHA was not significantly different in any N:P ratio however, the percent EPA was affected at a highest and lowest amount of KH₂PO₄. Conditions with the lowest KH₂PO₄ (N:P=15.95, 14.26 and 12.38) had the lowest total consumption of glucose.

Based on results at day 10, there is trend of lower N:P with higher percent fat. The N:P=9.85 obtained the highest percent fat overall, 64.4% with 11% EPA and 52.9% DHA and 5.93 DHA+EPA titer.

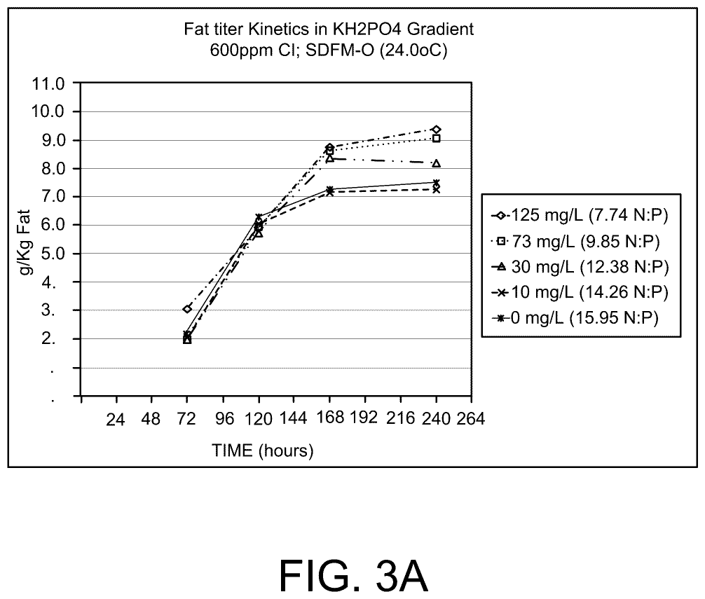

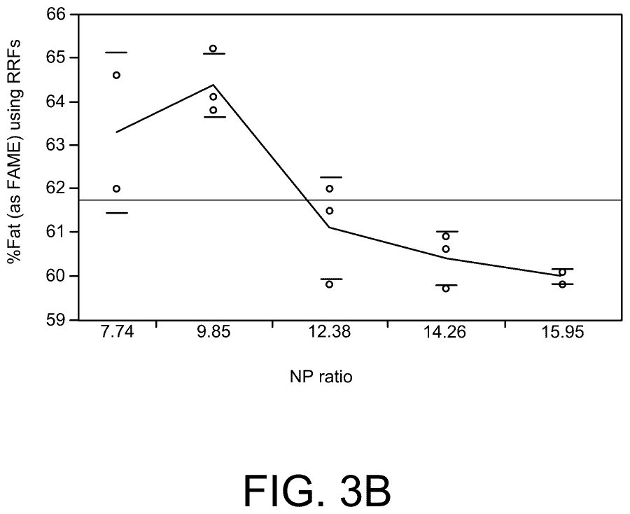

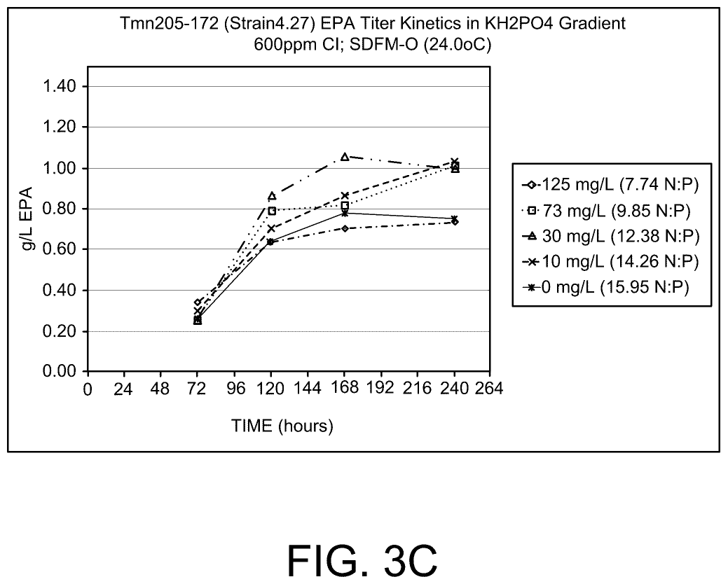

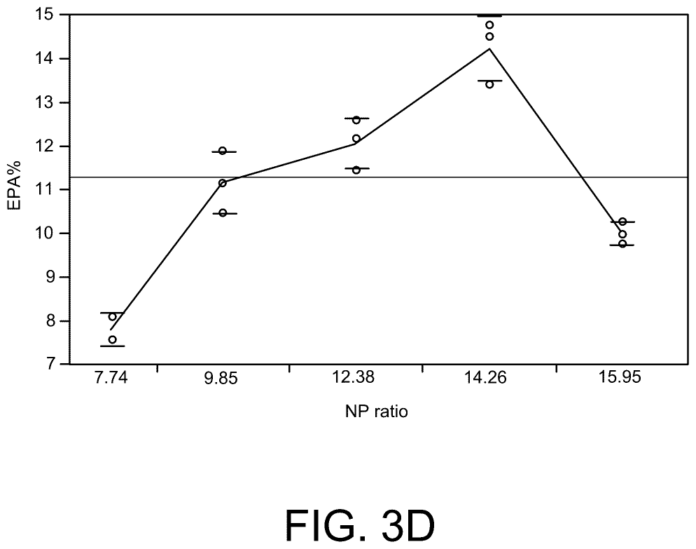

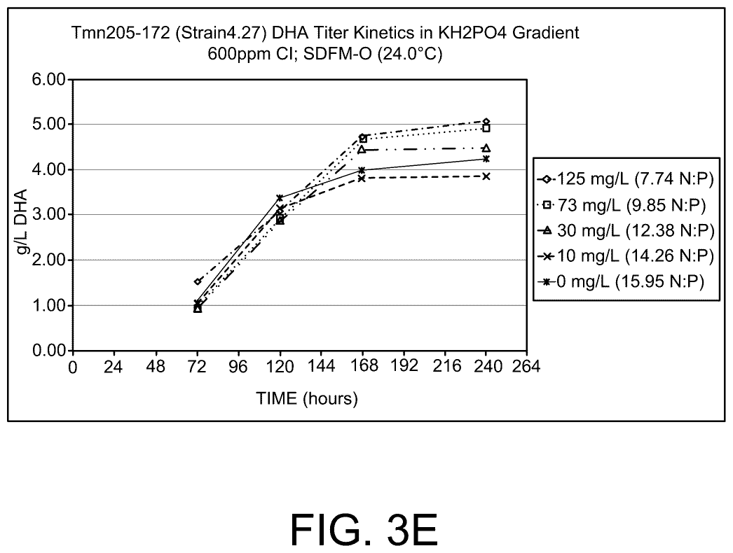

The fat titers and percent fat at day 10 seen in FIGS. 3A and 3B, respectively, show that the levels of each condition along with standard deviations shows a significant difference between N:P of 9.85 and 12.38. This difference indicates the impact of KH₂PO₄ concentration on the lipid production phase, as there is no impact on lean fat biomass (LFBM) in any of the N:P ratios tested. One possible explanation is that KH₂PO₄ affects the levels of malic enzyme (ME), glucose-6-phosphate dehydrogenase (GD6PDH), and isocitrate dehydrogenase activity, which has been shown to influence fatty acid synthesis for Schizochytrium sp HX-308 from early to late-stage fermentation.

The condition with no KH₂PO₄ batched (N:P of 15.95) had >45 g/Kg LFBM, which is similar to the other ratios, but exhibited no increased lipid production.

The EPA was also different in the N:P ratio gradient. The highest concentration of KH₂PO₄ (N:P=7.74) had an effect on the lipid profile, the amount of saturates (16:0) is increased and percent EPA is decreased significantly. This suggests that higher amounts of KH₂PO₄ may influence the lipid production profile in this strain. Also, the lowest KH₂PO₄ concentration (N:P=15.95) also showed a decrease in percent EPA but this is most likely due to the lower total fat production in the higher N:P ratio conditions.

The titer and percent EPA increased significantly at an N:P ratio of about 14.26 (FIGS. 3C and 3D, respectively) at day 10.

The titers of DHA did not change significantly in the three lower N:P ratios (7.74, 9.85 and 12.38) and only slightly in the higher ratios (14.26 and 15.95), which was likely due to the lower total fat production of these conditions FIG. 3E.

The glucose concentration/consumption rate obtained from this experiment was during the lipid production phase as the concentration of NH₄ and PO₄ was zero by the first harvest point LH72. Assuming, that lipid production starts after nitrogen limitation, the consumption rate was significantly reduced by LH168 for the conditions with the highest N:P (lower KH₂PO₄). The lower level of batched KH₂PO₄ had an effect in the ability of the cells to metabolize all the glucose available. Lower N:P of 7.74 and 9.85 conditions consumed most of the sugar and generated more biomass. However, LFBM was similar in all conditions (˜5 g/L). This may indicate that KH2PO4 has more of an impact during lipid production phase.

### Example 3

Experiments were performed to evaluate the addition of nitrogen during the production phase compared to a control without additional nitrogen feeding. Without being limited, it is hypothesized that adding nitrogen sources such as ammonium sulfate or glutamate during the production phase can regenerate the NADPH pool and maintain a higher productivity of fat and PUFA during the late lipid production phase.

Fermentations were performed as described in Example 1 using Schizochytrium, except either ammonium sulfate or monosodium glutamate were added at 10 mM (0.36 g/L ammonium ions) or 20 mM (2.9 g/L glutamate), respectively, at day 5, after the exhaustion of the batched nitrogen and phosphate in the media.

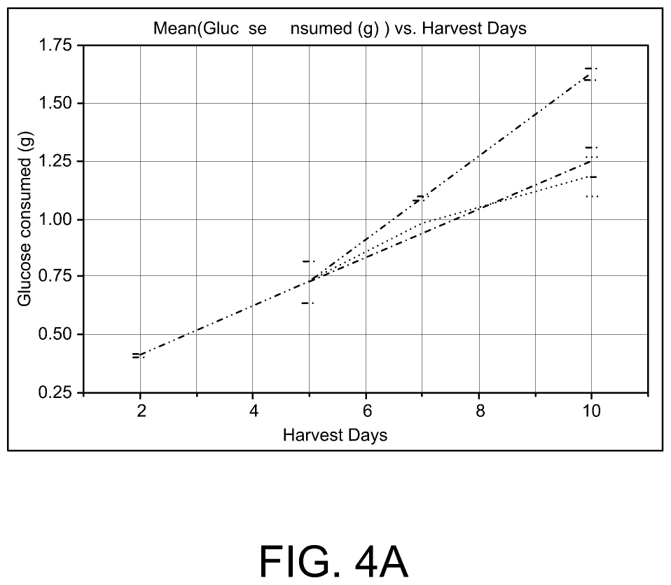

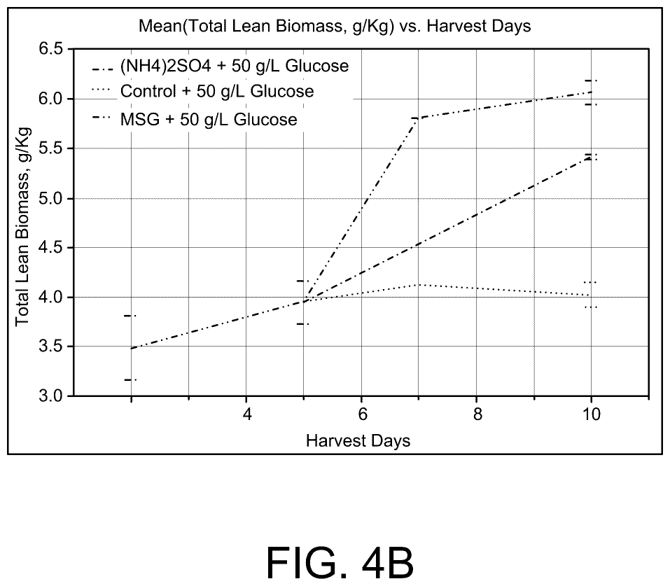

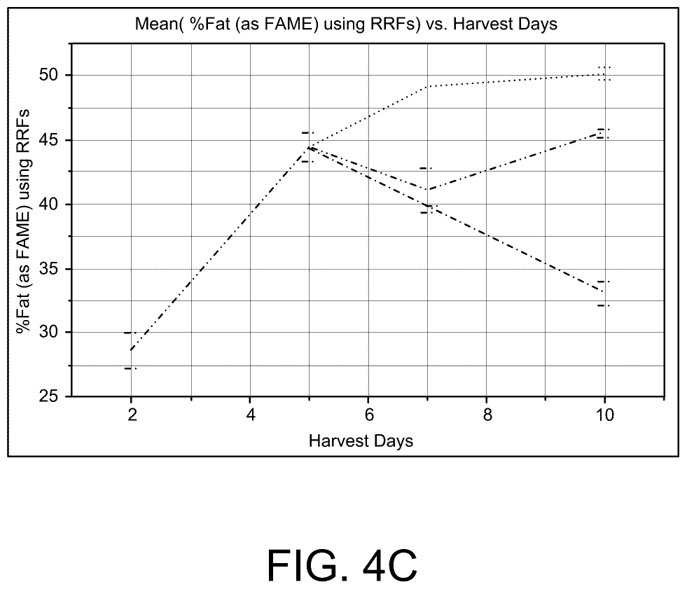

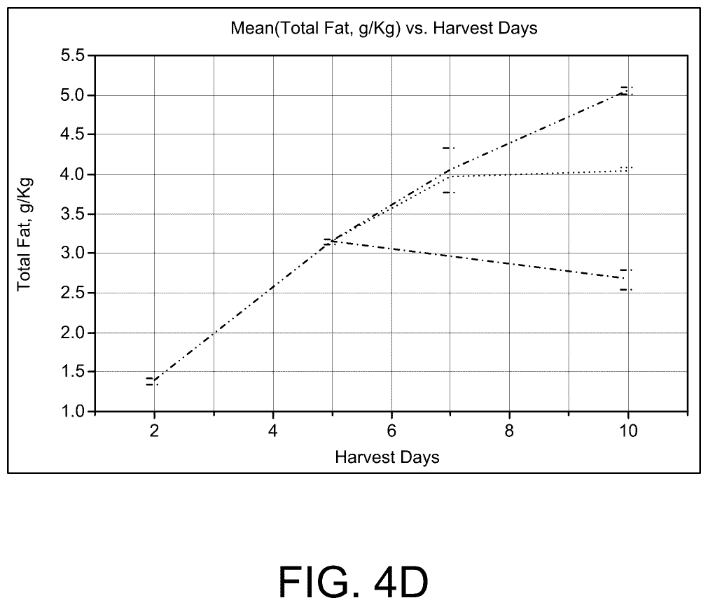

Glucose consumption (FIG. 4A): The addition of ammonium sulfate did not significantly impact glucose consumption as compared to a standard fermentation, whereas the addition of monosodium glutamate (top line) increased glucose consumption compared to a standard fermentation from about 1.25 g to 1.6 g after 10 days. See FIG. 4A comparing glucose consumption in the nitrogen fed vs. standard fermentation.

Impact on lean biomass (FIG. 4B): Addition of both nitrogen sources resulted in an increase in lean biomass. The increase in LFBM was significantly higher and faster for glutamate (top line) than ammonium sulfate (middle line) after 10 days.

Fat content (FIG. 4C): The addition of nitrogen (irrespective of the type) decreased the percent fat content as determined by FAME. The decrease in fat content was higher for the ammonium sulfate addition (bottom line).

Fat titer (FIG. 4D): Despite the lower total fat percent, the addition of glutamate and ammonium sulfate had opposite effects on the fat titer (g/kg). Glutamate (top line) increased fat titer compared to the control (middle line) while ammonium sulfate (bottom line) decreased fat titer compared to the control.

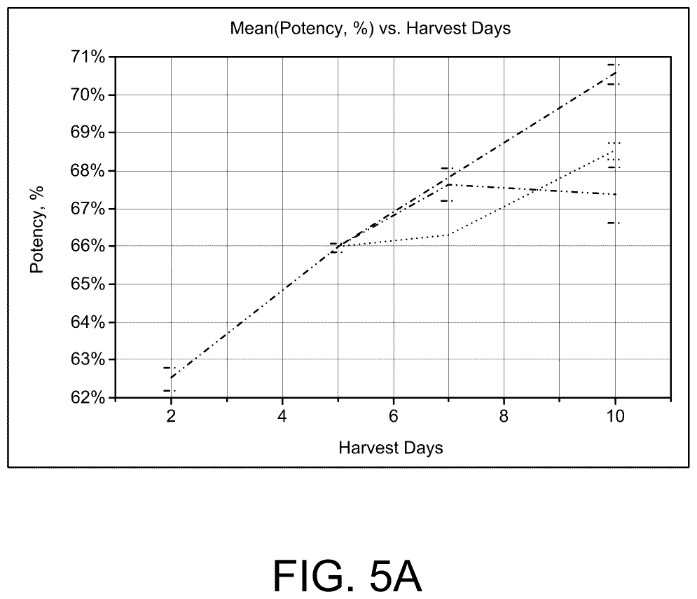

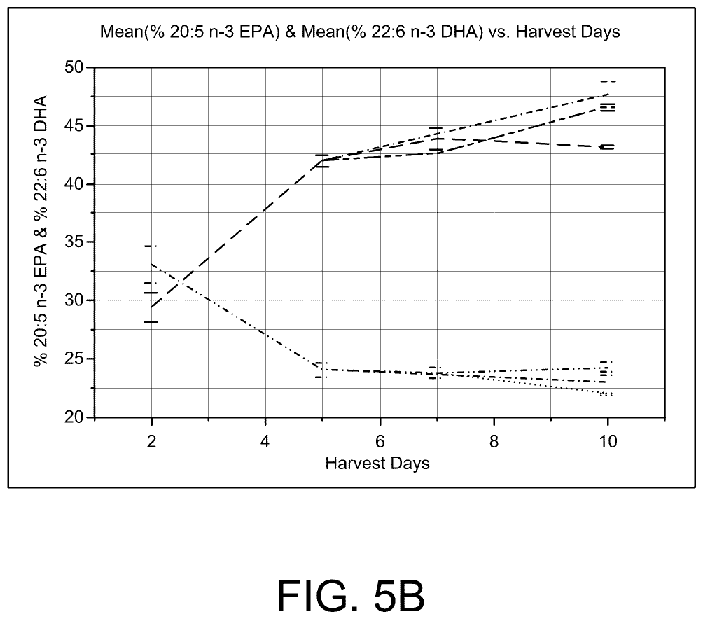

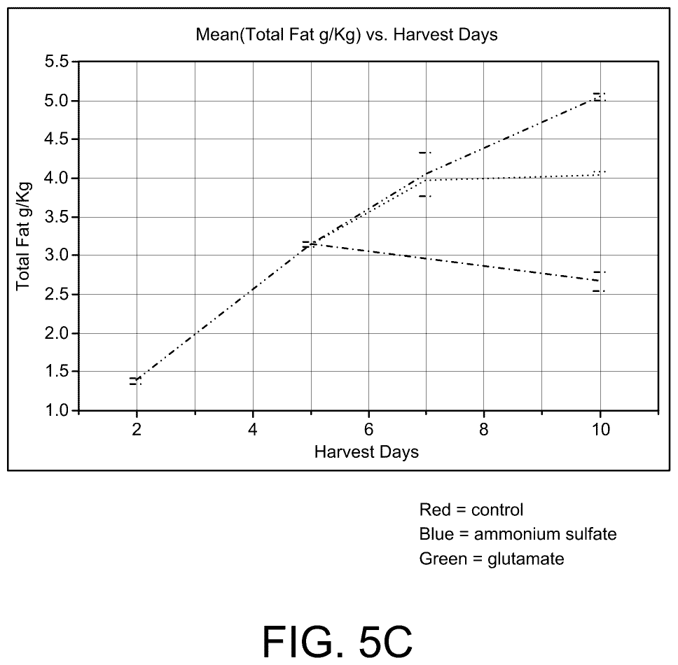

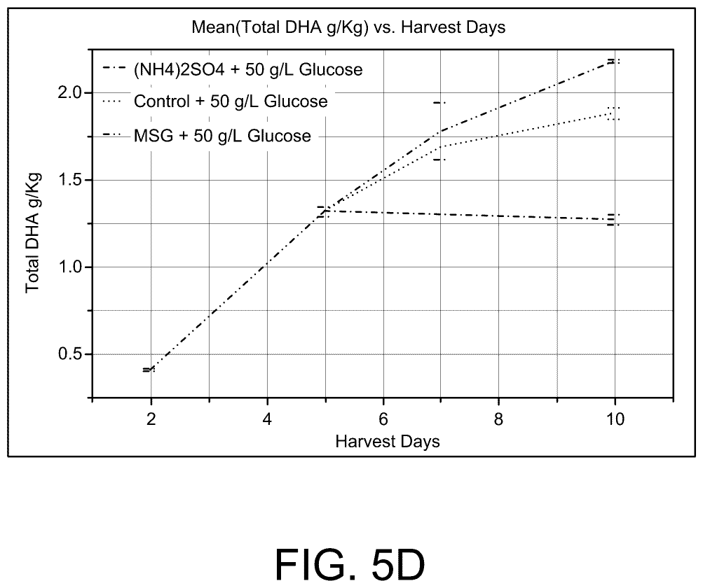

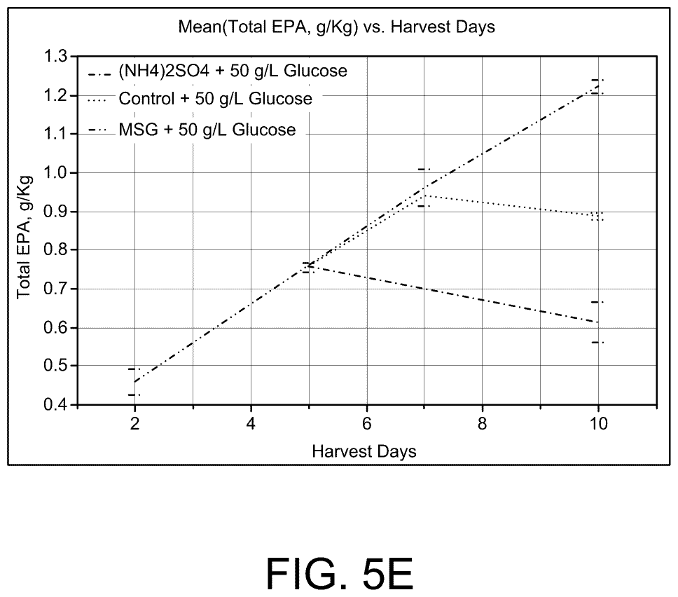

Potency (FIG. 5A): While the addition of ammonium sulfate decreased the total fat content, it increased the PUFA potency (top line, i.e., percent of PUFAs relative to total lipids). Glutamate had the opposite effect on potency after 10 days (bottom line). Overall, the changes were within few percentage points.

DHA & EPA composition (FIG. 5B): The percentages of DHA and EPA remained similar to the control with both nitrogen sources, with more DHA (dotted lines) and less EPA (solid lines) after 10 days.

PUFA Titer (FIGS. 5C-5D): The addition of glutamate also produced an increase in g/kg DHA (FIG. 5C, top line) and EPA (FIG. 5D, top line) with respect to control. Ammonium sulfate decreased the PUFA potency although it decreased the total fat content.

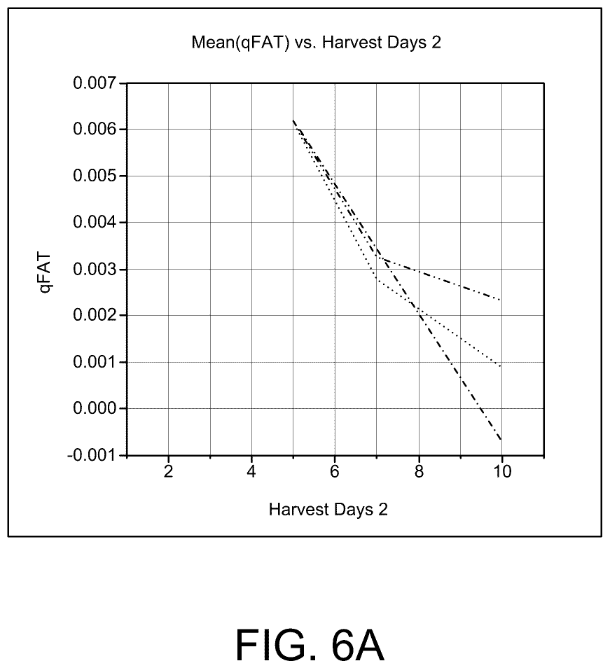

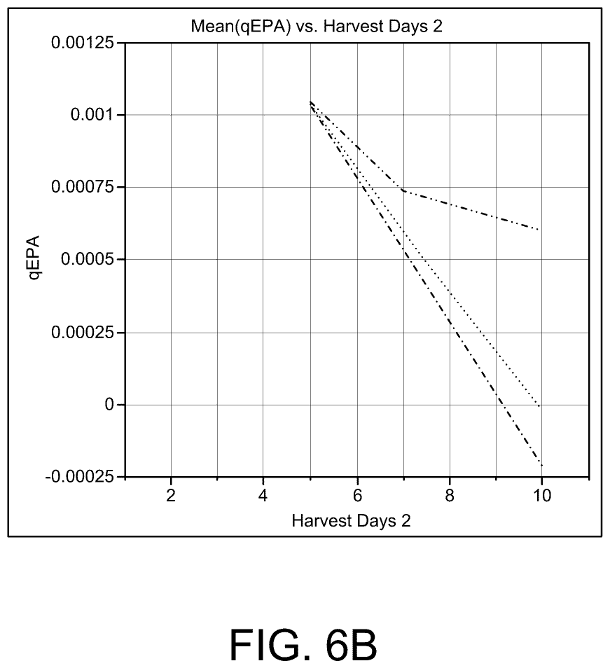

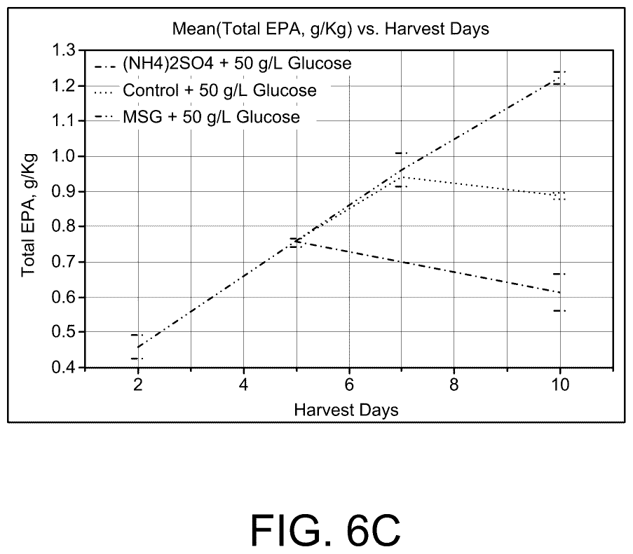

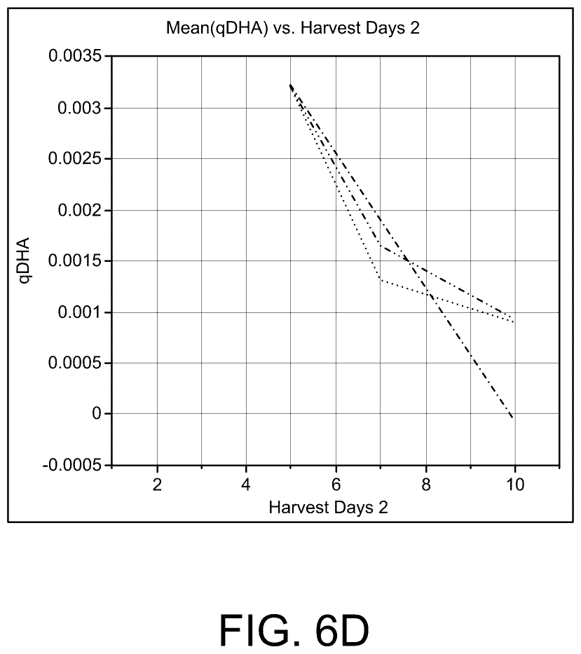

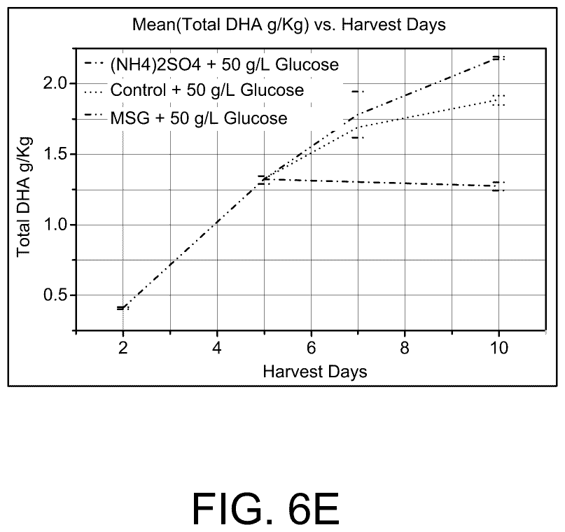

q-Rates. The q-rates for DHA, EPA and fat were calculated as g of product produced by g of cells over t-hours (g-product/(g-cells*hr)). Addition of glutamate increased the overall fat titer (FIG. 5, above) and this increase was driven by a corresponding increase in the mean q-Fat (FIG. 6A, top line) in comparison to the control (middle line).

The effect of glutamate on increasing the DHA and EPA titer can also be explained with a similar increase in corresponding qRates. Glutamate had the most drastic impact on improving the qRate of EPA (FIG. 6B and 6C) and maintains a higher qEPA between day 7 and day 10. The addition of glutamate resulted in an increase in fat and PUFA titer but also an increase in lean biomass that resulted in lowering the yield on glucose. Without limiting the invention, it is hypothesized that addition of nitrogen during the production phase can regenerate the NADPH pool and maintain a higher productivity PUFAs during the mid-to late lipid production phases The q-rate of DHA did not increase (FIG. 6D) but total DHA (g/kg) also increased with ammonium sulfate compared to control (FIG. 6E).

The addition of ammonium at a concentration of 0.36 g/L (10 mM) triggered growth at the expense of lipid production which dropped the yield substantially lower. This result was surprising because historically, ammonia levels below 0.1 g/Lin the 10 L during growth phase has resulted in stalling of growth.

All references disclosed are herein incorporated by reference in their entireties.

What is claimed:

1. A method of increasing production of eicosapentaenoic acid (EPA) in a microorganism, comprising increasing potassium or decreasing sodium in the growth and fermentation medium to lower the sodium to potassium ratio.

2. The method of claim 1, wherein the potassium is potassium sulfate.

3. The method of claim 1 or claim 2, wherein the sodium to potassium ratio is between about 1.85 to about 3.5.

4. The method of claim 3, wherein the sodium to potassium ratio is between about 2 to 3.

5. The method of any preceding claim, wherein the percentage of eicosapentaenoic acid as a percent of total lipids is increased by about 8-20% compared to standard fermentation medium with a higher sodium to potassium ratio.

6. The method of any preceding claim, wherein the microorganism is of the order Thraustochytriales.

7. The method of claim 6, wherein the species of Thraustochytriales is Schizochytrium.

8. A method of increasing lipid production in a microorganism, comprising decreasing the amount of phosphorus to increase the nitrogen to phosphorus ratio in the growth and fermentation medium.

9. The method of claim 8, wherein the phosphorus is provided as potassium phosphate.

10. The method of claim 8 or claim 9, wherein the lipids are polyunsaturated fatty acids.

11. The method of any one of claims 8-10, wherein a nitrogen to phosphorus ratio of between about 7 to about 13, optionally about 10, increases the percentage of lipids produced and/or increases the lipid titer.

12. The method of any one of claims 8-11, wherein a nitrogen to phosphorus ratio of between about 9.5 and 15, optionally between about 14-15, increases the percentage of eicosapentaenoic acid produced as a percent of total lipids.

13. The method of claim 12, wherein the EPA percentage is increased by at least about 5%.

14. The method of any one of claims 8-13, wherein the microorganism is of the order Thraustochytriales.

15. The method of claim 14, wherein the species of Thraustochytriales is Schizochytrium or Thraustochytrium.

16. A method of increasing the production of polyunsaturated fatty acids in a microorganism, comprising adding nitrogen during the lipid production (fermentation) phase.

17. The method of claim 16, wherein the nitrogen is glutamate, optionally, monosodium glutamate, or ammonium sulfate.

18. The method of claim 16 or claim 17, wherein the nitrogen is glutamate.

19. The method of claim 18, wherein the glutamate is added at about 3.0 g/L.

20. The method of any one of claims 16-19, wherein the polyunsaturated fatty acid is EPA and/or docosahexaenoic acid (DHA).

21. The method of claim 20, wherein the EPA and/or DHA is increased by at least 25%.

22. The method of any one of claims 16-21, wherein the nitrogen is ammonium sulfate.

23. The method of any one of claims 16-22, wherein the increased production is an increase in polyunsaturated fatty acid titer.

24. The method of any of claims 1-23, wherein the ratio of EPA to DHA is from about 0.5:1 to 2:1.

25. The method of any of claims 1-23, wherein the ratio of EPA to DHA is from about 0.5:1 to 1:1.
